(19) United States
(12) Patent Application Publication
(10) Pub. No.: US 2013/0164379 A1
(43) Pub. Date: Jun. 27, 2013

(54) NANOPARTICLE MICELLE COATED COMPOSITIONS

(71) Applicants: Andrei L. Gartel, Chicago, IL (US); Ming Wang, Tokyo (JP)

(72) Inventors: Andrei L. Gartel, Chicago, IL (US); Ming Wang, Tokyo (JP)

(21) Appl. No.: 13/668,075

(22) Filed: Nov. 2, 2012

Related U.S. Application Data
(60) Provisional application No. 61/555,110, filed on Nov. 3, 2011.

(51) Int. Cl. A61K 31/69; A61K 38/12

(52) U.S. Cl. CPC A61K 31/69; A61K 38/12
USPC 424/490; 514/19.9

(57) ABSTRACT

Nanoparticle micelle coated compositions and methods for treating a subject having a solid tumor are disclosed. The compositions comprise thiostrepton and a micelle-forming lipid wherein the thiostrepton is encapsulated inside a nanoparticle comprising the micelle-forming lipid. Pharmaceutical compositions comprising a thiostrepton-micelle composition are also disclosed.

### CROSS-REFERENCE TO RELATED APPLICATIONS

This application claims benefit of priority under 35 U.S.C. 119 to U.S. provisional patent application Ser. No. 61/555,110, filed Nov. 3, 2011, and entitled “Nanoparticle Micelle Coated Compositions,” the contents of which are herein incorporated by reference in its entirety.

### STATEMENT OF GOVERNMENT INTEREST

This invention was made with Government support under grant numbers R01CA129414, 1R21CA134615 and 4564256542642 awarded by the National Institutes of Health. The Government has certain rights in the invention.

### FIELD OF INVENTION

This invention relates to compositions for cancer treatment.

### BACKGROUND

Cancer poses a significant cause of mortality in the United States that requires improved therapeutic strategies. Many promising anti-cancer drugs are limited by physiochemical properties that affect their efficacy in administration and bioavailability. For example, many anti-cancer compounds are hydrophobic, rendering them insoluble in aqueous solutions that are commonly used for administration.

One promising anti-cancer agent is the thiazole antibiotic thiostrepton. This compound has been shown to inhibit cell growth in a variety of human cancer cell lines. The apoptotic activity of thiostrepton is attributed in part to inhibiting proteasome activity, resulting in stabilizing certain proteins that are fatal to cancer cells. Yet other targets like the forkhead box M1 transcription factor FOXM1, which are often over-expressed in cancer cells, are suppressed by proteasome inhibitors like thiostrepton.

Because thiostrepton is hydrophobic, challenges remain in how clinicians administer the drug to patients at a level adequate for bioavailability and efficacy. While thiostrepton analogs having improved aqueous solubility properties might be feasible, challenges may persist whereby such analogs are bioconverted to active species. Furthermore, the controlled, selective biodistribution of soluble analogs or converted active species of thiostrepton to intended sites of pharmacological action remains problematic. Isolated solubilized small molecules like thiostrepton can enter a variety of otherwise healthy organs that are not subject to disease or that harbor tumors. Not only does this misdirected delivery pose a threat to healthy tissues and organs, but it also reduces the effective concentration of pharmacologically active species to tumors.

Thus, there is a need for compositions and methods that enable one to selectively deliver thiostrepton to appropriate target tumors and at a therapeutically effective concentration for achieving anticancer effect.

### SUMMARY

In one respect, the invention relates to a composition for treating a subject having a solid tumor. The composition includes thiostrepton and a micelle-forming lipid. The thiostrepton is encapsulated inside a nanoparticle comprising the micelle-forming lipid.

In a second respect, the invention relates to a method for treating a subject having a solid tumor. The method includes the step of administering to said subject a nanoparticle micelle coated composition of thiostrepton.

In a third respect, the invention relates to a pharmaceutical composition for treating a subject having a solid tumor. The pharmaceutical composition includes a thiostrepton-micelle composition.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1a depicts a thiostrepton and a preferred PEG-lipid (DSPE-PEG₂₀₀₀-MeO).

FIG. 1b depicts a preferred nanoparticle micelle coated composition that includes thiostrepton surrounded by a micelle layer composed of a preferred PEG-lipid (DSPE-PEG₂₀₀₀-MeO).

FIG. 1c depicts the extent of loading thiostrepton as a function of the ratio of thiostrepton:lipid.

FIG. 2a illustrates physicochemical properties of preferred thiostrepton-micelle compositions disclosed herein, such as the particle size distribution (left panel); particle surface charge as reflected by Zeta potential (right panel).

FIG. 2b depicts an electron micrograph image of the micelles alone (left panel) and the resultant micelle-encapsulated thiostrepton particles (right panel).

FIG. 2c illustrates thiostrepton release profiles from the micelle-encapsulated thiostrepton particle composition.

FIG. 3a depicts caspase-3 cleavage profiles for HepG2-luc cells treated without and with thiostrepton-micelle compositions.

FIG. 3b depicts caspase-3 cleavage profiles for MDA-MB-231 cells treated without and with thiostrepton-micelle compositions.

FIG. 3c depicts cell viability assays for HepG2-luc cells treated without and with thiostrepton-micelle compositions.

FIG. 3d depicts cell viability assays for MDA-MB-231 cells treated without and with thiostrepton-micelle compositions.

FIG. 4a depicts cell fluorescence from tumors following administration of fluorescently-tagged thiostrepton-micelle compositions.

FIG. 4b illustrates the percentage of injected dose for free thiostrepton and micelle coated thiostrepton that remains following 4 hr and 24 hr post-injection.

FIG. 4c illustrates the tumor volume growth over time for animals receiving no treatment, micelle treatment or micelle coated thiostrepton treatment.

FIG. 4d illustrates final tumor masses from animals receiving no treatment or micelle coated thiostrepton treatment.

FIG. 4e depicts tumors obtained from animals receiving no treatment or micelle coated thiostrepton treatment.

FIG. 5a illustrates the results of HepG2-luc liver cancers from animals, wherein animals treated with thiostrepton-micelle compositions had reduced tumor volumes compared with untreated animals.

FIG. 5b illustrates the results of HepG2-luc liver cancers from animals, wherein animals treated with thiostrepton-micelle compositions had reduced final tumor masses compared with untreated animals.

FIG. 5c illustrates the sizes of HepG2-luc liver cancers from animals receiving no treatment or treatment with thiostrepton-micelle compositions.

FIG. 6a depicts biochemical analyses of protein expression of HepG2-luc liver cancers from animals with respect to caspase-3 expression and FOXM1 levels as a function of whether the animals received the thiostrepton-micelle compositions.

FIG. 6b depicts cytological analyses of protein expression of HepG2-luc liver cancers from animals with respect to caspase-3 expression and FOXM1 levels as a function of whether the animals received the thiostrepton-micelle compositions.

FIG. 7a depicts results of combination treatment of thiostrepton and bortezomib in inducing apoptosis in tumor cells in vitro for osteosarcoma cell line, U2OS-C3.

FIG. 7b depicts results of combination treatment of thiostrepton and bortezomib in inducing apoptosis in tumor cells in vitro for pancreatic cancer cell line, MiaPaca-2.

FIG. 7c depicts results of combination treatment of thiostrepton and bortezomib in inducing apoptosis in tumor cells in vitro for ovarian cancer cell line, PA-1.

FIG. 7d depicts results of combination treatment of thiostrepton and bortezomib in inducing apoptosis in tumor cells in vitro for colon cancer cell line, HCT-116 (wt and FOXM1-knockdown lines).

FIG. 7e depicts results of combination treatment of thiostrepton and bortezomib in inducing apoptosis in tumor cells in vitro for breast cancer cell line, MDA-MB231 (wt and FOXM1-knockdown lines).

FIG. 8a depicts results of combination treatment of thiostrepton and bortezomib in inducing cell death in tumor cells in vitro for colon cancer cell line, HCT-116 using sub-apoptotic concentrations of thiostrepton, bortezomib and thiostrepton/bortezomib combinations.

FIG. 8b depicts results of combination treatment of thiostrepton and bortezomib in inducing cell death in tumor cells in vitro for breast cancer cell line, MDA-MB231 using sub-apoptotic concentrations of thiostrepton, bortezomib and thiostrepton/bortezomib combinations.

FIG. 8c depicts results of combination treatment of thiostrepton and bortezomib in inducing cell death in tumor cells in vitro for pancreatic cancer cell line, MiaPaca-2 using sub-apoptotic concentrations of thiostrepton, bortezomib and thiostrepton/bortezomib combinations.

FIG. 9a depicts results of combination of thiostrepton and bortezomib in inhibiting cell proliferation in tumor cells in vitro for colon cancer cell line, HCT-116, where the combination index chart for the drug combination is plotted with CI on the y-axis and fractional effect (FI) on the x-axis.

FIG. 9b depicts results of combination of thiostrepton and bortezomib in inhibiting cell proliferation in tumor cells in vitro for pancreatic cancer cell line, MiaPaca-2, where the combination index chart for the drug combination is plotted with CI on the y-axis and fractional effect (FI) on the x-axis.

FIG. 9c depicts results of combination of thiostrepton and bortezomib in inhibiting cell proliferation in tumor cells in vitro for breast cancer cell line, MDA-MB231, where the combination index chart for the drug combination is plotted with CI on the y-axis and fractional effect (FI) on the x-axis.

FIG. 9d depicts results of combination of thiostrepton and bortezomib in inhibiting cell proliferation in tumor cells in vitro for ovarian cancer cell line, PA-1, where the combination index chart for the drug combination is plotted with CI on the y-axis and fractional effect (FI) on the x-axis.

FIG. 10a depicts clonogenic assay results showing the long-term effect of combination treatment of thiostrepton and bortezomib in vitro on the colon cancer cell line, HCT-116.

FIG. 10b depicts clonogenic assay results showing the long-term effect of combination treatment of thiostrepton and bortezomib in vitro on the breast cancer cell line, MDA-MB231.

FIG. 11a depicts organ biodistribution profile from animals treated with thiostrepton-micelle compositions, bortezomib, or a combination of thiostrepton-micelle compositions and bortezomib.

FIG. 11b depicts tumor size from animals treated with thiostrepton-micelle compositions, bortezomib, or a combination of thiostrepton-micelle compositions and bortezomib.

FIG. 11c depicts tumor volume from animals treated with thiostrepton-micelle compositions, bortezomib, or a combination of thiostrepton-micelle compositions and bortezomib.

FIG. 11d depicts whole animal weight from animals treated with thiostrepton-micelle compositions, bortezomib, or a combination of thiostrepton-micelle compositions and bortezomib.

FIG. 12a illustrates immunohistochemical profiles for tumors isolated from animals untreated and treated with a combination of thiostrepton-micelle compositions and bortezomib with regard to caspase-3 cleavage.

FIG. 12b illustrates immunohistochemical profiles for tumors isolated from animals untreated and treated with a combination of thiostrepton-micelle compositions and bortezomib with regard to PARP expression.

FIG. 13a illustrates proteasome activity isolated from tumors from animals treated with a combination of thiostrepton-micelle compositions and bortezomib.

FIG. 13b illustrates the activity of thiostrepton and bortezomib on purified proteasomes.

FIG. 14a illustrates the uptake of fluorescently-tagged thiostrepton-micelle compositions into tumors of treated animals.

FIG. 14b illustrates animal autopsy for untreated and treated animals.

FIG. 14c depicts tumor profiles for untreated and treated animals.

FIG. 14d depicts body weights for untreated and treated animals.

### DETAILED DESCRIPTION OF THE INVENTION

Applicants have developed robust nanoparticle micelle coated compositions for encapsulation of thiostrepton that greatly enhances its solubility and optimizes its tumor-associated biodistributional profile upon administration. The nanoparticle micelle compositions are amphiphilic in that they possess a hydrophilic outer shell and a hydrophobic inner core in which the hydrophobic thiostrepton drug can be solubilized. The nanoparticle micelle encapsulation acts not only as a means to solubilize thiostrepton but also aids in delivering the thiostrepton to tumors with high specificity as compared to healthy tissues. Unlike the vasculature structure of healthy, non-cancerous tissues, the neovasculature that supplies blood flow to tumors is highly irregular and punctuated with fenestrations. These junctions provide openings with diameters of up to about 700 nm, wherein particles having a size less than about 700 nm in diameter can leave the circulation only via diffusion through the fenestrated gaps of tumor blood vessels. Finally, the nanoparticle micelle coated compositions of thiostrepton are amenable for use in co-administration regimens, thereby providing utility for multidrug-based combination chemotherapies.

### Nanoparticle Micelle Coated Compositions

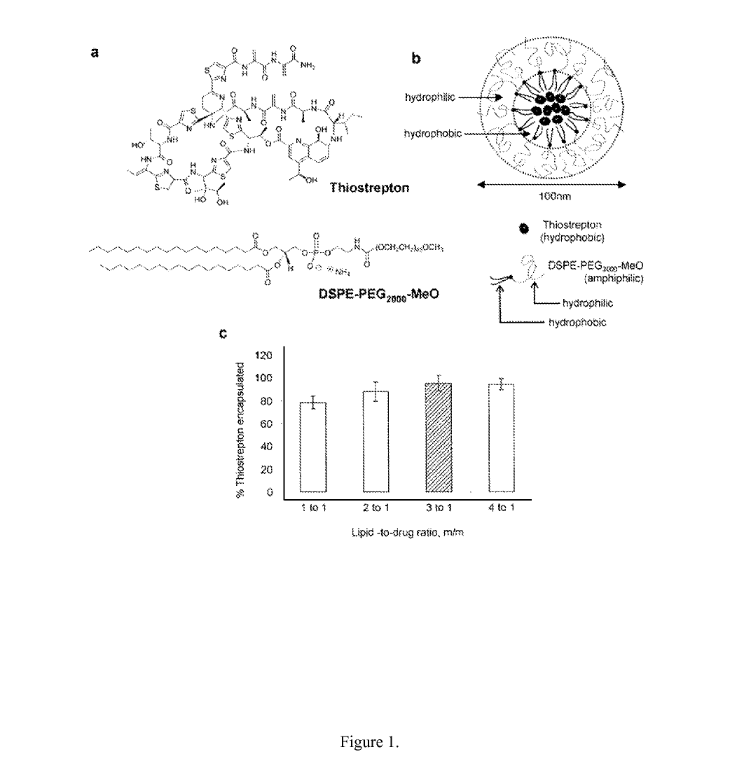

FIG. 1 illustrates the components used to assemble the nanoparticle micelle coated compositions. FIG. 1a depicts the chemical structures of thiostrepton and a preferred lipid, which is 1,2-distearoyl-sn-glycero-3-phosphoethanol-amine-N-[methoxy(polyethylene glycol)-2000] (DSPE-PEG₂₀₀₀-MeO), which can be used for forming the micelle. A preferred nanoparticle micelle coated composition includes a plurality of thiostrepton molecules encapsulated by a plurality of DSPE-PEG₂₀₀₀-MeO molecules (FIG. 1b). Such nanoparticle micelle coated compositions can be preferably formed with the lipid-hydration method using a suitable solvent (for example, chloroform). The encapsulation conditions are optimized by varying the lipid:thiostrepton molar ratio from about 1:1 to about 4:1 (molecule:molecule), wherein a highly preferred lipid:thiostrepton molar ratio of about 3:1 is achieved with DSPE-PEG₂₀₀₀-MeO as the preferred lipid component (FIG. 1c). The preferred concentration of thiostrepton ranges from about 0.2 mM to about 10.0 mM, with a highly preferred concentration of thiostrepton being within the range from about 1.0 mM to about 3.0 mM. The preferred concentration of DSPE-PEG₂₀₀₀-MeO is above its critical micelle concentration, with a highly preferred concentration of DSPE-PEG₂₀₀₀-MeO being about 3-fold that of the corresponding concentration of thiostrepton.

The DSPE-PEG₂₀₀₀-MeO represents a preferred polymer-lipid conjugate owing to its ability to spontaneously form micelles with hydrophobic cores upon dispersion in aqueous solutions. PEGylated lipids, as exemplified by DSPE-PEG₂₀₀₀-MeO, are amphiphilic lipid-polymer conjugates where the polymer component is a hydrophilic polymer chain such as polyethylene glycol (PEG). Upon hydration above their respective critical micelle concentrations, these lipid-PEG conjugates form amphiphilic micelle structures consisting of a hydrophilic polymer shell, and an organic lipidic core. It is within this lipid-rich area that thiostrepton can be solubilized and ultimately incorporated into nano-sized macromolecular structures that can aid its accumulation into tumor sites.

Key physicochemical attributes of such nanoparticle micelle coated compositions are that they form nanoparticle micelle coated compositions having a diameter less than about 700 nm and displaying negative Zeta potentials. The size attribute of the nanoparticle is an important physicochemical property since the compositions are designed to diffuse passively through fenestrated gaps in tumor blood vessel, wherein the gaps have a diameter of up to about 700 nm. The negative Zeta potential attribute of the nanoparticle is an important physicochemical property since a negatively-charged nanoparticle surface would prevent opsonin recognition, thereby preventing macrophage-assisted clearance of the nanoparticle from the circulation due to opsonization.

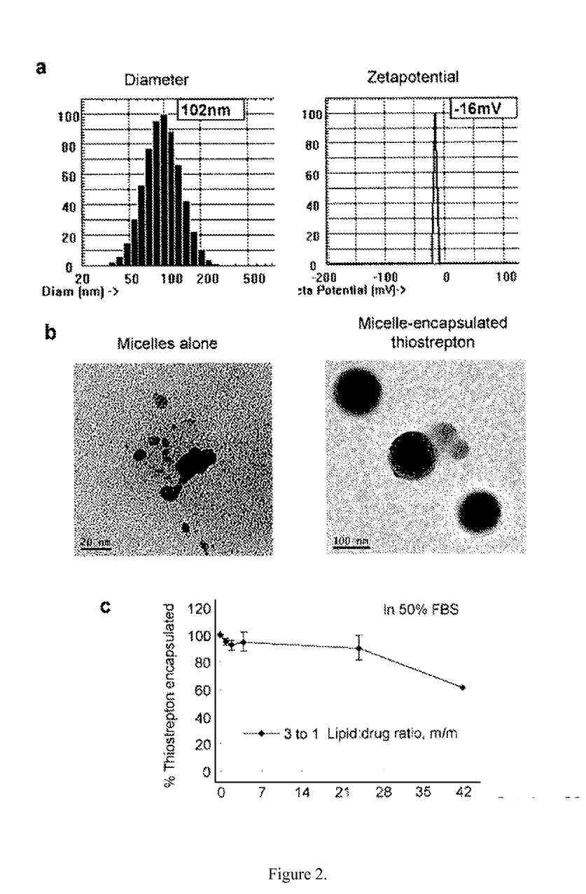

For the preferred nanoparticle micelle coated compositions composed of the DSPE-PEG₂₀₀₀-MeO as the micelle forming agent, the compositions have a average diameter of ˜100 nm (FIG. 2a, left) and a Zeta potential of about −16 mV (FIG. 2a, right). As demonstrated in the Examples, these compositions have robust utility as chemotherapeutic agents in treating solid tumors with exquisite specificity and a preferred biodistributional profile.

One of ordinary skill in the art would recognize based upon this disclosure that micelle forming agents at a concentration above their respective critical micelle concentrations that form nanoparticles having the aforementioned physicochemical properties (for example, a nanoparticle having a diameter less than about 700 nn and a negative nanoparticle surface Zeta potential) would be amenable for use to form nanoparticle micelle coated compositions of the present invention. Table I provides other examples of commercially available lipids amenable for use in forming the nanoparticle micelle coated compositions of the present invention.

TABLE I
Lipids suitable for the nanoparticle micelle coated compositions¹

1,2-distearoyl-sn-glycero-3-phosphoethanol-amine-N-
[methoxy(polyethylene glycol)-X] (X = 1000 MW;
2000 MW 5000 MW; 10000 MW; 20000 MW; or 40000 MW)
1,2-distearoyl-sn-glycero-3-phosphoethanol-amine-N-
[amino(polyethylene glycol)-X]
(X = 1000 MW; 2000 MW; 3400 MW; 5000 MW; or 10000 MW)
1,2-distearoyl-sn-glycero-3-phosphoethanol-amine-N-
[maleimide(polyethylene glycol)-X]
(X = 1000 MW; 2000 MW; 3400 MW; 5000 MW; or 10000 MW)
1,2-distearoyl-sn-glycero-3-phosphoethanol-amine-N-
[hydroxyl(polyethylene glycol)-X] (X = 5000 MW)
1,2-distearoyl-sn-glycero-3-phosphoethanol-amine-N-
[azido(polyethylene glycol)-X] (X = 3400 MW; or 5000 MW)
1,2-distearoyl-sn-glycero-3-phosphoethanol-amine-N-
[silane(polyethylene glycol)-X] (X = 3400 MW)
1,2-distearoyl-sn-glycero-3-phosphoethanol-amine-N-
[succinyl(polyethylene glycol)-X] (X = 2000 MW)
1,2-distearoyl-sn-glycero-3-phosphoethanol-amine-N-
[carboxyl(polyethylene glycol)-X] (X = 2000 MW)
Poly(lactic-co-glycolic-acid) (PLGA), polyethyleneglycol (PEG)
Poly(ethylene oxide)-Poly(α-benzyl L-aspartate)
Poly(ethylene oxide)-block-poly(L-aspartate)
Poly(ethylene oxide)-β-p(ε-caprolactone)
Polyethylene Glycol-Diacyl lipid
Hydrolyzed polymer of epoxidized soybean oil
¹The “-X” designation refers to the molecular masses of the PEG component of the lipid.
Combination chemotherapeutic compositions with nanoparticle micelle coated compositions of thiostrepton and another chemotherapeutic agent having efficacy for treating solid tumors and liver tumors.

The nanoparticle micelle coated compositions of thiostrepton are robust for use in combination with a non-encapsulated chemotherapeutic agent. For example, bortezomib, another proteasome inhibitor having a different specificity relative to thiostrepton, can bring about a complementary reduction in solid tumors and tumors associated with liver when administered in combination with nanoparticle micelle coated compositions of thiostrepton disclosed herein (see Examples).

Thus, one of ordinary skill in the art would recognize from this disclosure that the nanoparticle micelle coated compositions of thiostrepton have tremendous utility as a component of multidrug-based combination chemotherapies for treating a variety of liver and solid tumors wherein the co-administered drugs can be administered in free form (that is, non-encapsulated form) or administered in a nanoparticle micelle coated compositional form, like that of thiostrepton. Optionally, hydrophobic drugs like thiostrepton may be co-administered a mixed nanoparticle micelle coated compositions containing both the thiostrepton and the other hydrophobic drug agent. One of ordinary skill in the art would recognize from this disclosure that such complex nanoparticle micelle coated compositions containing thiostrepton and another hydrophobic drug agent can be assembled in pharmacologically optimal nanoparticle micelle coated compositions in the same manner as formed for compositions containing only thiostrepton as the active chemotherapeutic agent using the same procedures described herein.

### Administration Routes and Applicable Treatments

The nanoparticle micelle coated compositions described herein are designed for biodistribution to liver and solid tumors by diffusion via fenestrated gaps within the blood vessels. Thus, intravenous administration routes are a preferred means of administering the compositions.

Tumors of the liver and solid tumors of other organs generally are the preferred targets for applicable treatments. Solid tumors amenable to treatment with the compositions disclosed herein include any tumor having a neovasculature comprising fenestrated gaps with a diameter sufficient to enable diffusion of the nanoparticles from the blood vessels to the target tumor tissue.

The compositions disclosed herein can be implemented for treating solid tumors from any biological tissue. As used herein, “source of biological tissue” refers to organs, tissues, samples, extracts, biopsies, explants, implants, transplants, grafts, engineered biologically compatible materials, and the like, which reside within a biological organism, which can be adapted for physiological or biophysical purpose within a biological organism, or which can be obtained from a biological organism, whether living or dead. As used herein, “type of biological tissue” refers to biological tissues from particular organs and tissue systems having physiological functions and, with respect to mammals, include adrenal gland, anus, bladder, brain, breast, blood vessels, bone, cardiac tissue, cardiovascular system tissues, colon, connective tissue, epithelial tissue, esophagus, eye, gall bladder, heart, intestine, kidneys, larynx, liver, lung, mouth, muscle, neck, nervous system tissue (for example, spinal cord), nose, olfactory, pancreas, parathyroid gland, pineal gland, pituitary gland, prostate, sexual organ tissues (for examples, cervix, vagina, penis, etc.), sinus, skin, spleen, stomach, throat, tongue, thyroid gland, rectum, renal system tissues, reproductive tissues (for examples, ovaries, uterus and testicles), among others.

Preferably, “biological organism” refers to a plant or animal composed of at least one tissue and/or organ system in a differentiated and/or undifferentiated form (for example, containing stem cells and/or terminally differentiated cells, respectively) and in a normal and/or non-normal form (for example, healthy vs. microbially-infected, diseased, or cancerous states). More preferably, biological organism refers to an animal (for examples, amphibians, birds, fish, mammals, reptiles, and invertebrates) having a differentiated, multiple organ physiology and metabolism system. Most preferably, biological organism refers to a mammalian animal, such as a human, dog, cat, hamster, mouse, monkey, rat, pig, cow, horse, among others. Human is a highly preferred mammal for use with the compositions disclosed herein. Examples provide illustrations of the use of the compositions for treating cancers in animal model systems.

### EXAMPLES

### Example 1

### Preparation and Characterization of Nanoparticle Micelle Coated Compositions Containing Thiostrepton

### Preparation of Nanoparticle Micelle Coated Compositions Containing Thiostrepton

A thin lipid-film of DSPE-PEG₂₀₀₀-MeO and thiostrepton was prepared by mixing the two components in chloroform in a 10 mL round-bottomed flask. The chloroform was removed in vacuo and by air-drying, after which the lipid film was hydrated (for final thiostrepton concentration of 1 mM or 2 mM) with PBS (pH 7) or H₂O and vortexed at room temperature for 10 min. The lipid-to-drug ratio was varied by keeping the amount of thiostrepton constant (at 1 mM) and increasing the amount of PEG-lipid from 1 mM to 4 mM. The percentage of encapsulated thiostrepton was measured by centrifuging solutions of micelle-thiostrepton (varying lipid-to-drug ratios) at 8 RCF for 2 min, after which the supernatant was separated from any precipitated matter (insoluble, unencapsulated thiostrepton). The supernatant was measured for UV absorbance at λ=300 nm, from which the amount of remaining encapsulated thiostrepton was calculated. To measure the amount of non-encapsulated thiostrepton, the precipitates were dissolved in CHCl₃ and examined for UV absorbance at λ=300 nm. For micelle-only preparations, the thiostrepton component was omitted and only the lipid component was hydrated in aqueous solution to 3 mM or 6 mM, corresponding to either 1 mM thiostrepton or 2 mM thiostrepton. For fluorescence labeling, DOPE-Rhodamine was incorporated at the chloroform stage at a 1:100 molar ratio to DSPE-PEG₂₀₀₀-MeO.

### Characterization of Nanoparticle Micelle Coated Compositions Containing Thiostrepton

For drug release studies, micelle-thiostrepton suspensions (3:1 or 4:1 lipid/drug ratios m/m) were incubated in 50% FBS (in PBS, final concentrations of 0.05 mM thiostrepton) over time at 37° C. Amount of remaining encapsulated thiostrepton was measured by centrifuging incubated samples (8 RCF, 2 min) to remove the non-encapsulated and precipitated thiostrepton from the micelle-encapsulated thiostrepton in the supernatant. The supernatant was then measured for UV absorbance at λ=300 nm and the concentration of remaining thiostrepton was calculated by extrapolation with calibration curves. Sizes and Zeta potentials of nanoparticles (in PBS) were analyzed by dynamic light scattering, using a 5 mW 633 nm laser angled at 90° to the sample. Measurements are presented as volume-weighted multimodal distributions. For in vitro and in vivo applications, micelle-encapsulated thiostrepton solutions were prepared in PBS (sterile) at 1 mM or 2 mM, respectively. For transmission electron microscopy, 20 μL of micelle-thiostrepton solutions (1 mM thiostrepton/3 mM lipid) or micelle-only solutions (3 mM lipid) in H₂O were dispensed onto a 200-mesh Cu grid supported by holey-carbon film and air-dried overnight. TEM images were collected using the JEOL JEM-3010, equipped with a LaB6 electron gun operated at 300 kV. Images were captured using a 1K×1K Peltier-cooled Gatan multi-scan CCD camera.

Assembled micelle-thiostrepton structures were found to be in the form of nanoparticle structures with hydrodynamic diameter of 100 nm and a surface Zeta potential of −16 mV (FIG. 2a). Transmission electron microscopy images illustrated the difference in micelles before and after drug encapsulation and confirm the presence of 100 nm-diameter micelle-thiostrepton species (FIG. 2b). The release profile of the optimal formulation were examined by incubating micelle-thiostrepton nanoparticles (3:1 lipid/drug, m/m) in PBS solutions containing 50% FBS at 37° C., to mimic the plasma serum conditions of in vivo blood circulation (FIG. 2c). In 50% FBS, the integrity of the micelle-thiostrepton structure was maintained for long periods of time, where 90% of thiostrepton was maintained within nanoparticle structures after 24 hr of incubation. This finding is particularly significant for the in vivo application of such nanoparticles, where the maintenance of drug encapsulation in circulation for longer periods is required for accumulation into tumor sites.

### Example 2

Cell Culture Studies In Vitro with Nanoparticle Micelle Coated Compositions of Thiostrepton
Cell cultures and treatment regimens.

MDA-MB-231 (MDA-MB-231-luc-D3H2-LN, human lymph node-derived mammary gland adenocarcinoma, Caliper Lifescience) cells were seeded at 1×10⁵ cells per 10-cm plate and HepG2-luc cells were seeded at 5×10⁵ cells per 6-cm plate and incubated overnight before treatment. Non-encapsulated thiostrepton (dissolved in DMSO, 10 mM) and micelle-encapsulated thiostrepton (1 mM) were administered to cells at concentrations of 5 μM, 7.5 μM and

10 μM. Controls for in vitro experiments were cells treated with either DMSO only (6 μL) or micelle only (30 μM). Cells were treated for 24 hr before harvesting.

### Western Blot Analysis of Cell Lysates

Cells were harvested with IP lysis buffer (20 mM HEPES, 1% Triton X-100, 150 mM NaCl, 1 mM EDTA, 1 mM EGTA, 100 mM NaF, 10 mM Na₄P₂O₇, 1 mM Na₃VO₄, 0.2 mM PMSF). For analysis of tissues, liquid N₂-frozen sections of xenograft tumors were homogenized in 1 mL IP lysis buffer. Protein concentration was measured by the Bio-Rad Protein Assay and protein separation was performed on 8% or 12% SDS-PAGE gels. Separated proteins were then transferred onto PVDF membranes (Millipore) and immunoblotted with specific antibodies against FOXM1 (c-20, Santa Cruz), cleaved caspase-3 (Cell signaling) and β-actin (Sigma). Quantification of cleaved caspase-3 and FOXM1 protein expression levels was performed by the ‘Gel’ analysis function in ImageJ.

### Cell Viability Assay

Cells were seeded at a density of 1000 cells per well into 96-well plates. After incubation overnight, growth media was replaced with media containing free (in DMSO) or micelle-encapsulated thiostrepton at increasing concentrations. DMSO-treated and micelle-only-treated cells were also examined and used to compare with non-treated (media only) cells. Cells were treated for 72 hr before media was spiked with 3-(4,5-dimethylthia-zo1,2-yl)-2,5-diphenyltetrazolium bromide (MTT) assay reagent (Sigma, prepared in 5 mg/mL in PBS) to render a final 10% concentration. After incubation for 3 hr, MTT/media was removed and purple precipitates were dissolved with 150 μL of DMSO. Wells were measured for absorbance at λ=550 nm and the % cell viability was calculated as a percentage of the UV absorbance of non-treated cells.

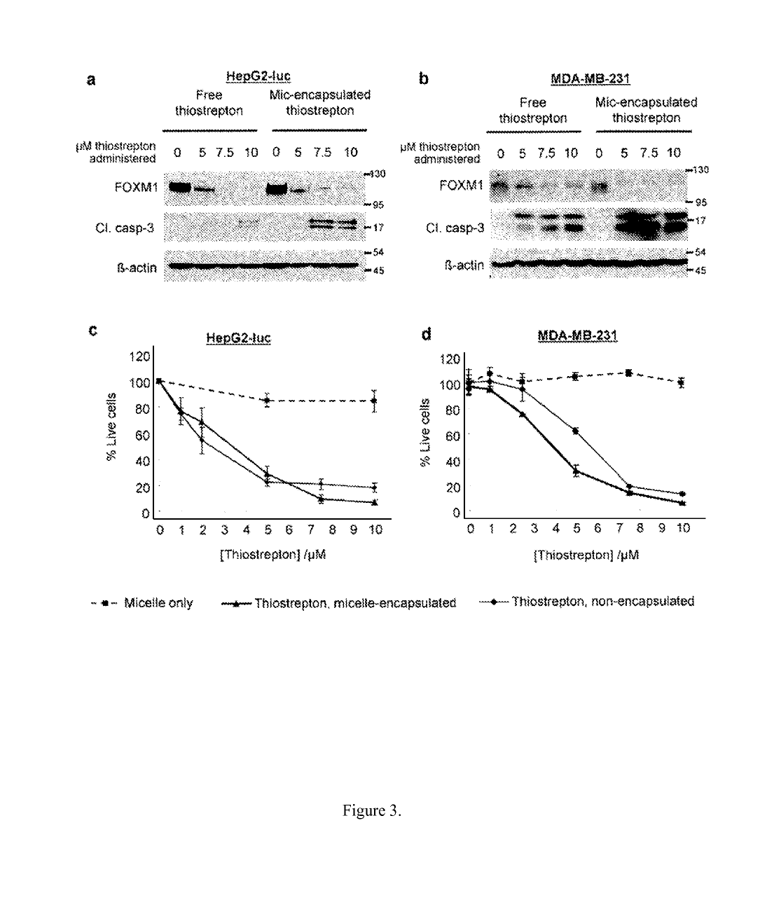

Treatment of HepG2-luc (luciferase-expressing) liver cancer cells and MDA-MB-231 breast cancer cells and with micelle-encapsulated thiostrepton resulted in an enhancement of cleaved caspase-3 expression (a marker of apoptosis), compared to those treated with non-encapsulated thiostrepton (FIGS. 3a and 3b). Also observed is the further suppression of FOXM1, in cells treated with micelle-encapsulated thiostrepton, compared to free thiostrepton. As thiostrepton is highly insoluble (precipitates of DMSO-dissolved thiostrepton are highly visible when added to cell media), it is likely that its full encapsulation into micelles increases its availability to cancer cells in vitro. Cell viability assays also confirmed the enhanced effect of thiostrepton, when delivered to cells through micelle-encapsulation (FIGS. 3c and 3d). In terms of mechanism of drug internalization within the cancer cells, it is likely that thiostrepton is released into the cell media before it is internalized into cancer cells, as incubation with fluorescently-labeled micelle-thiostrepton did not show cell-associated fluorescence over time. As for empty micelles, they alone did not have an effect on cell viability, suggesting their low toxicity for in vivo applications.

### Example 3

Animal Tumor Model Studies In Vivo with Nanoparticle Micelle Coated Compositions of Thiostrepton

### Animal Maintenance and Tumor Xenograft Experiments

Animals were maintained and treated in accordance with the guidelines established by the Animal Care and Use Committee of UIC. Tumor models were prepared by implanting cancer cell lines (1×10⁶ of MDA-MB-231 and 2×10⁶ of HepG2-luc), suspended in 50 uL of 1:1 PBS/Matrigel into each flank of 4-week old male athymic mice (Taconic). Treatment began once tumors reached sizes of 30 mm³ or 150 mm³. After completion of the dosing schedule, animals were sacrificed and tumors were removed. Tumors were sliced in half and either frozen in liquid N₂ or fixed overnight in 10% formalin (4° C.).

### Biodistribution of Micelle-Thiostrepton Nanoparticles

Rho-labeled micelle-thiostrepton nanoparticles were injected intravenously via the tail-vein into non-tumor-bearing or MDA-MB-231 tumor-bearing animals (1 cm³ per tumor) at a dose of 1.8 mg thiostrepton per animal At 4 hr, 17 hr and 24 hr post-injection, animals were anaesthetized with isoflurane and imaged using the Xenogen IVIS system for rhodamine-fluorescence distribution. Animals were then sacrificed and organs removed for further fluorescence imaging. Background autofluorescence was eliminated using a spectral unmixing algorithm provided by Living Image software. For comparison with free, non-micelle-encapsulated drug, thiostrepton was dissolved to concentrations of 36 mg/mL in a N,N-dimethylacetamide/polyethylene glycol/Tween 80 formulation (2:7:1) and administered at a dose of 50 μL per animal (2 animals, total 4 tumors) by i.p. injection. Tumors were collected at 4 hr and 24 hr post-injection and were homogenized (Fisher, Polytron) in 1 mL lysis buffer. Tumor homogenates were treated with 200 μL 10mM HCl for 24 hr at 4° C. One milliliter of chloroform was then added and the homogenates were vortexed for 10 min. Samples were incubated at room temperature for 24 hr before vortexing, after which samples were centrifuged and the chloroform supernatant layer was collected and dried in vacuo. For HPLC/Mass spectrometry studies, extracted samples were reconstituted in 50 μL of chloroform and 450 μL of MeOH. Using an Agilent Zorbax 300sb-C8, 2.1×50 mm column, the isocratic method of 30% of 100% H₂O/0.1% Formic acid and 70% of 100% Methanol/0.1% formic acid at 200 μl/ml for 6 min was run. The AUC of HPLC traces that correlated to thiostrepton retention times were used to calculate the concentrations of thiostrepton, as compared to a thiostrepton/HPLC calibration curve.

Treatment of Xenograft Models with Micelle-Encapsulated Thiostrepton

Animals bearing tumors were randomized into groups of 4-5, where groups were administered with micelle-only controls at 200 mg DSPE-PEG₂₀₀₀-MeO/kg, (200-250 μL of 6 mM DSPE-PEG₂₀₀₀-MeO in PBS) or 30-40 mg/kg of thiostrepton encapsulated in micelles (2 mM thiostrepton/6 mM _{DSPE-PEG}₂₀₀₀-MeO in PBS, 200-300 μL). Treatments were performed once every two days (3 times a week), during which tumor volumes were monitored with calipers (l×w×h=volume, mm³). In the MDA-MB-231 model, injections were carried out daily for the last 5 days. Animals were also weighed once a week. After 12-14 treatments, animals were sacrificed and tumors removed.

### Immunohistochemical Analysis of Paraffin-Embedded Tissue

Formalin-fixed tumor tissues were embedded in paraffin and sliced to 4 μm-thick sections. Sections were deparaffinzed by submerging sample-containing slides into a series of solvents of decreasing hydrophobicity. Heating samples in 10 mM citric acid (pH 6) was used for antigen retrieval. After blocking with 3% H₂O₂ in MeOH, and further blocking in normal goat serum in PBS, slides were treated with primary anti-FOXM1 (Santa Cruz, k-19), anti-cleaved caspase-3 or control IgG antibodies at concentrations of 1:100, in 1% BSA/PBS. Biotinylated secondary antibody treatment and Avidin HPR treatment was carried out according to the Vectastain ABC kit manual (anti-rabbit, Vector Labs), followed by staining with DAB (Sigma, D-0426). Cleaved caspase-3 stained samples were counterstained with hematoxylin (Vector Labs). Samples were dehydrated in the series of solvents and mounted with Permount (Fisher) mounting agent. Slides were analyzed on a Zeiss Apotome microscope.

Accumulation of Micelle-Encapsulated Thiostrepton into MDA-MB-231 Breast Cancer Xenografts and its Effect on Tumor Growth

Nano-sized particles can localize specifically into tumor sites by bypassing healthy tissue and diffusing through the leaky fenestrations in the tumor blood vessels, a vascular characteristic shared with no other organ apart from the liver. Further to their specific localization, nano-sized structures are retained in tumor sites due to the impaired lymphatic drainage system of tumor tissue. The ability of administered micelle-thiostrepton to localize into tumors was investigated using nude mice bearing MBA-MB-231 xenografts. The tumor retention and biodistribution of micelle-thiostrepton was studied by LC/MS of the tumor homogenates and by live animal fluorescence imaging.

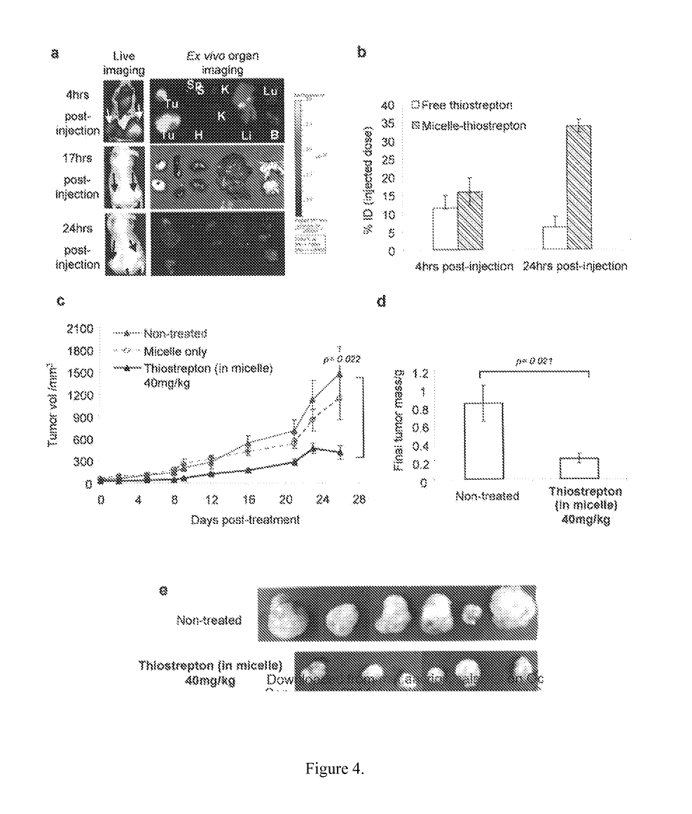

Upon establishment of subcutaneous tumors, fluorescently-labeled micelle-thiostrepton was administered through the tail vein and animals were monitored for rhodamine-associated fluorescence by live whole body imaging (Xenogen IVIS). Accumulation of fluorescence into tumor sites (live and ex vivo imaging) was observed to occur to a maximum at 4 hr post-administration (FIG. 4a). Localization into the liver was also prominent at 4 hr post-administration, likely an event of diffusion through the sinusoidal capillaries, which are also punctuated with fenestrations as it is in angiogenic tumor vessels. By 17 hr, micelle-associated fluorescence had greatly diminished in tumor regions, although a small amount appeared retained. As fluorescence labeling only monitors the movement and retention of the lipid moiety of the micelle-thiostrepton complex, tumor homogenates were further extracted with chloroform (in which thiostrepton is soluble) and the amount of tumor-accumulated thiostrepton was examined using LC/MS. It was found that tumor-associated thiostrepton was in fact in higher concentrations at 24 hr post-injection, compared to that at 4 hr post-injection (FIG. 4b). In conjunction with the fluorescence-biodistribution data, this is indicating that micelle-thiostrepton complexes arrive at tumor sites intact, and that it is also likely that thiostrepton continues to accumulate into tumors 4 hr post-administration (up until 17 hr), after which dissociation of the micelle-thiostrepton occurs and the lipid component is cleared from the region. Furthermore, the percentage ID (injected dose) of micelle-thiostrepton to arrive at tumors was approximately 30% per tumor, and considering there were two xenograft tumors per animal, 60% of the ID was tumor-localized (FIG. 4b). We compared the tumor accumulation of micelle-thiostrepton with that of its solubilization into the previously reported N,N-dimethylacetamide/polyethylene glycol/Tween 80 formulation and found that micelle-encapsulated thiostrepton accumulated into tumors with greater efficiency, where an increase in approximately 10-fold of thiostrepton concentrations were detected in each tumor (FIG. 4b). In addition, pilot studies also showed the effect on xenograft growth of thiostrepton delivered through the dimethylacetamine-based formulation was much weaker in MDA-MB-231 tumors compared to delivery through micelle-encapsulation.

The biological effect of micelle-encapsulated thiostrepton on tumor growth was monitored in MDA-MB-231 subcutaneous tumor models. The specific dose for administration (40 mg/kg) was selected after pilot studies showed it to be the minimal concentration to exhibit anti-cancer effects. Injections were administered 3 times a week, which after 14 treatments, reduced tumor growth by up to 4-fold, compared to non-treated tumors (FIG. 4c). As determined from the release studies, thiostrepton is not likely released from the micelle vehicle before it reaches the tumor sites, as thiostrepton can remain encapsulated for at least 24 hr in high serum conditions. Internalization of whole rhodamine-labeled micelle-thiostrepton complexes were shown to be possible at higher serum conditions, as indicated by increase in cell associated fluorescence over time. Therefore, micelle-thiostrepton complexes likely arrive intact at tumor vicinities and are internalized into the tumor cells by mechanisms such as endocytosis. As with non-treated tumors, tumors treated with an equivalent dose of empty micelles (without thiostrepton) increased steadily in size over time, and a reduction in tumor growth rate was not observed (FIG. 4c). Final tumor weights of harvested tumors also correlated to tumor volume data, where non-treated tumors were on average 4 times heavier than micelle-thiostrepton-treated tumors (FIGS. 4d and 4e).

### Micelle-Encapsulated Thiostrepton Inhibits Tumor Growth in a HepG2-luc Liver Cancer Subcutaneous Xenograft Model

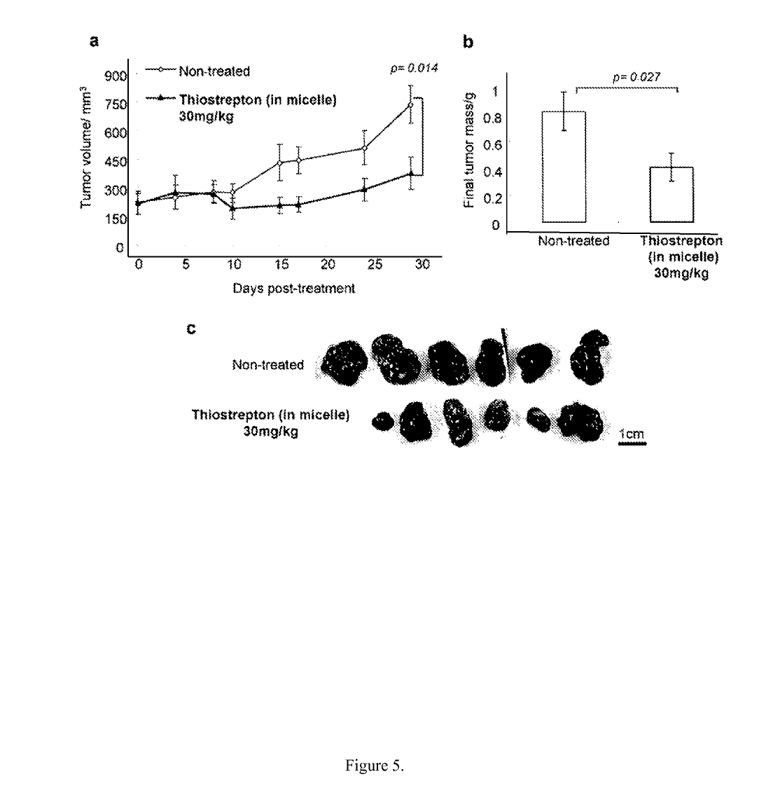

Subcutaneous HepG2-luc liver cancer cells were left to proliferate until the size of the liver cancer xenografts reached 200 mm³, as to examine the effect of thiostrepton treatment on already larger tumors. As in the previous tumor model, the accumulation of micelle-thiostrepton into HepG2-luc xenografts was proven using rhodamine-associated whole body imaging. Doses were then administered at 30 mg/kg, 3 times a week for 4 weeks and tumor progression was monitored by both caliper measurements and luciferase imaging. Again, the specific dose was chosen after pilot studies suggested it to be the minimum at which an effect can be observed in this model tumor. After completion of the dosing schedule, micelle-thiostrepton-treated tumors were found to be half the volume of the non-treated groups (FIG. 5a). The implanted HepG2-luc tumors also express luciferase, therefore, their change in luciferase expression during the dosing gave insight into tumor cell viability. Compared to that of the beginning of treatment, tumor-associated luciferase in micelle-thiostrepton-treated tumors was much less than that of non-treated tumors. After the treatment schedule, tumors were also removed and weighed, and treated tumors were found to be half the weight of non-treated tumors (FIGS. 5b and 5c).

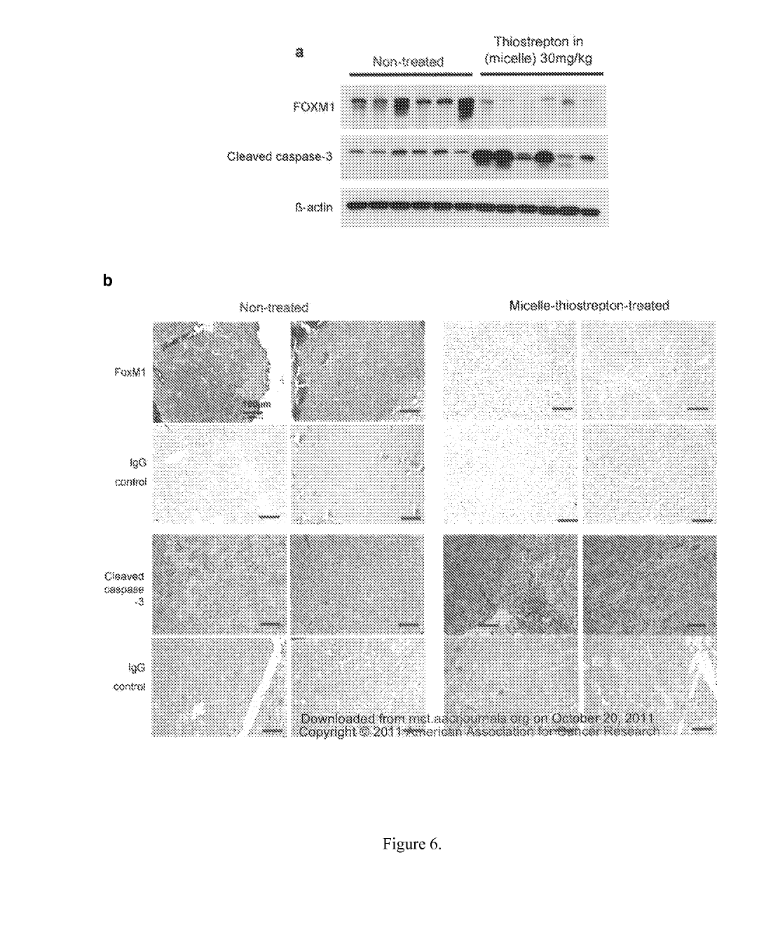

Removed tumors from xenograft models were then analyzed for markers of apoptosis by western blot and immunohistochemistry. Homogenized tumors showed overall an evident increase in the expression of cleaved-caspase-3, a marker of apoptosis (FIG. 6a). Furthermore, the treatment of in vivo cancers with thiostrepton, or any proteasome inhibitor leads to suppression of the cancer-associated transcription factor, FOXM1. Immunohistochemistry of tumor samples reinforce the effect found in homogenized tumors, where the expression of cleaved-caspase-3 is higher and FOXM1 levels are lower in micelle-treated tumors, compared to non-treated tumors (FIG. 6b).

### Example 4

Animal Tumor Model Studies In Vitro with Nanoparticle Micelle Coated Compositions of Thiostrepton in Combination with Bortezomib

### Cell Lines

U2OS-C3 osteosarcoma, PA-1 ovarian, HCT116 colon and MiaPaca-2 pancreatic cancer cells were cultured in DMEM medium (Invitrogen). HCT-116 and MiaPaca-2 (shFOXM1) cells were cultured in DMEM medium (Invitrogen). MDA-MB231 (parental and shFOXM1) cells were grown in RPMI-1640 medium (Invitrogen). The media were supplemented with 10% fetal bovine serum (Atlanta Biologicals) and 1% penicillin-streptomycin (GIBCO) and the cells were kept at 37° C. in 5% CO₂. Stable cell lines were generated by transduction of FOXM1 shRNA lentiviral particles (Sigma) followed by selection with puromycin (Sigma).

### Combination Index Assay

The effect of thiostrepton and bortezomib combination was determined by the MTT assay. 3-(4,5-Dimethylthiazol-2-yl)-2,5-diphenyltetrazolium bromide (MTT) was procured from Sigma (M5655). Cells were plated at a density of 1×10⁴ per well in 200 μL of complete culture medium and treated with thiostrepton alone, bortezomib alone and combination of thiostrepton and bortezomib in 96-well micro titer plates. After incubation for 72 hr at 37° C. in a humidified incubator, 10 μL MTT (5 mg/mL in PBS) was added to each well, following which the plate was centrifuged briefly. After careful removal of the medium, 0.1 mL buffered DMSO was added to each well. The absorbance was recorded on a micro-plate reader at the wavelength of 540 nm. In our experiments, the IC₃₀, IC₅₀, IC₇₀, IC₈₀, and IC₉₀ values (i.e., the drug concentration needed to cause 30%, 50%, 70%, 80%, and 90% reductions in cell viability) were chosen for comparison. CI values were measured by Chou-Talalay method.

### Detection of Apoptosis

The AnnexinV-PE staining kit (559763, BD Bio. Sci.) was used for the detection of apoptotic bodies following the vendor's protocol. This kit uses a dual-staining protocol in which the cells show fluorescence of Annexin V (apoptotic cells) and fluorescence of 7AAD (necrotic cells or late apoptotic cells). Briefly the tumor cells were grown at a density of 50% confluence in 100-mm culture dishes and were treated with varying concentrations of the drugs for 24 hr. The cells were trypsinized, washed with PBS, and were processed for labeling with annexinV-7AAD. The labeled cells were analyzed by flow cytometry.

### Immunoblotting

Actively dividing cells were seeded into a 100-mm plate at a density of 7.5×10⁵ cells. Cells were treated with bortezomib alone, thiostrepton alone and combination of thiostrepton and bortezomib for 24 hr following which the cells were lysed. Cells were lysed in IP buffer (20 mM HEPES, 1% Triton X-100, 150 mM NaCl, 1 mM EDTA, 1 mM EGTA, 100 mM NaF, 10 mM Na₄P₂O₇, 1 mM sodium orthovanadate, 0.2 mM PMSF supplemented with protease inhibitor tablet and the protein concentration was determined using the Bio-Rad protein assay reagent. Fifty micrograms of the cell lysates were separated by electrophoresis on SDS-polyacrylamide mini gel and transferred to PVDF membrane Immunoblotting was performed with specific antibodies for cleaved caspase-3 (9664 Cell Signaling), FOXM1 (Dr. Costa) and β-actin (A5441, Sigma).

### Nuclear-ID Green Chromatin Condensation Detection

Cells were stained using in vitro apoptosis detection kit (51021-K200 Enzo Life Sciences), according to the manufacturer's recommendations. Briefly, 3-4×10⁵ cells were plated in 60-mm culture dishes and allowed to grow overnight. Cells were treated with the sub-lethal concentrations of thiostrepton, bortezomib or thiostrepton methyl ester or combination of thiostrepton and bortezomib and thiostrepton-methyl ester and bortezomib. Following overnight incubation of drugs cells were trypsinized and submitted for flow cytometry analysis. Analysis was done by using FL-1 channel of flow cytometer with excitation wavelength of 488 nM. Staurosporine, provided in the kit was used as a positive control.

### Clonogenic Survival Assay

HCT-116 and MDA-MB231 cells were plated to medium plates at 3×10⁵ cells confluence and treated with combination of thiostrepton and bortezomib for 24 hr. The cells were then trypsinized, re-suspended in the media and counted. The cells were re-seeded (750 cells per medium plate) and incubated for 10 days. Fresh media was added on the fifth day. On the tenth day, media was removed from the dishes and washed once with ice-cold PBS. The colonies were stained with 2 ml each of 0.25% 1,9-dimethyl-methylene blue (341088, Sigma) in 50% ethanol for 45 minutes on a rocking platform. The dishes were rinsed three times with PBS and air-dried and scanned.

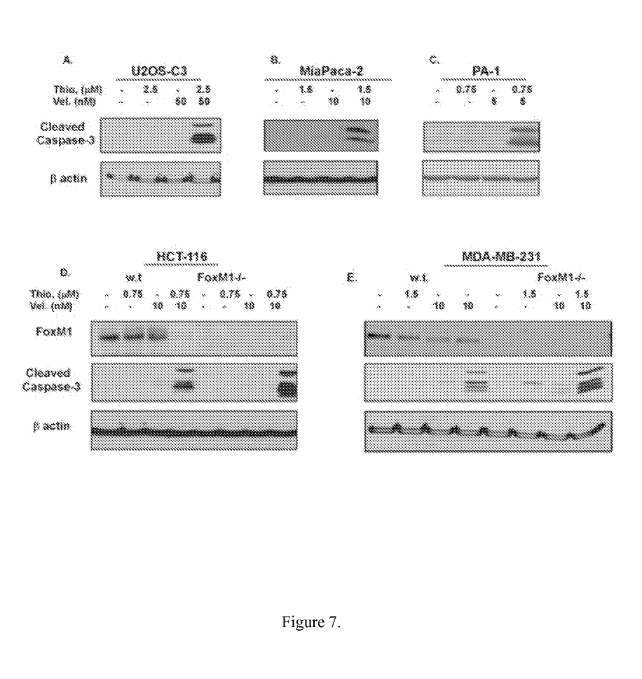

To determine whether thiostrepton may synergize with bortezomib against human cancer cell lines of different origin, osteosarcoma, pancreatic, and ovarian cancer cells were treated with either sub-apoptotic concentrations of thiostrepton or bortezomib alone or with combinations of the two agents for 24 hr and used caspase-3 to serve as an indicator of apoptotic cell death (FIG. 7a-7c). While treatment with thiostrepton or bortezomib alone induced little or no caspase-3 cleavage in these cells, treatment with combination of these drugs showed potent caspase-3 cleavage in U2OS-C3 osteosarcoma cells (FIG. 7a). Similar synergistic effects of thiostrepton/bortezomib combinations on caspase-3 cleavage were seen in MiaPaca-2 pancreatic cancer, PA-1 ovarian, HCT-116 colon and MDA-MB-231 breast cancer cells (FIG. 7b-7e).

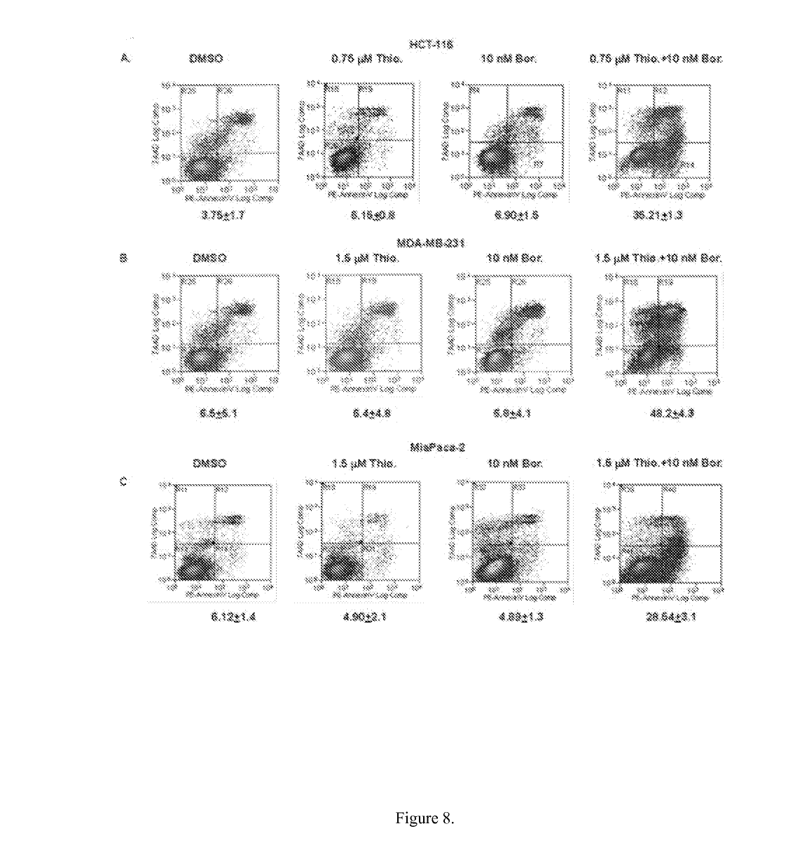

Following the demonstration of synergistic induction of cell death following co-treatment with thiostrepton and bortezomib, the effect of presence of FOXM1 in the apoptosis induced by thiostrepton and bortezomib combination was investigated on HCT-116 colon and MDA-MB231 breast wild-type (wt) and FOXM1 knockdown cancer cells. Tumor cells (wt and FOXM1 knockdown) were treated with sub-apoptotic concentrations of thiostrepton alone, bortezomib alone or combination of the two agents. In the wt cells, the bortezomib and thiostrepton combination demonstrated synergy in induction of cell death and down-regulation of FOXM1 following treatment. However, more importantly, knockdown of FOXM1 in tumor cells sensitized the tumor cells to thiostrepton and bortezomib combination (FIGS. 7d and 7e). To further demonstrate that combination treatment of thiostrepton and bortezomib induces synergistic apoptosis, we stained these cells (DMSO treated, thiostrepton treated, bortezomib treated and treated together with the two drugs) with annexin V-PE/7AAD and analyzed them by flow cytometry. As shown in FIG. 8a, treatment of HCT-116 cells with 0.75 μM thiostrepton or 10 nM bortezomib induced apoptosis of only 6.1% and 6.9% over the control, while treatment with both drugs at the same doses caused 35.2% of cells to undergo apoptosis. Similar synergistic effect of thiostrepton/bortezomib combination was observed by annexin-VPE-7AAD staining in MDA-MB-231 breast and MiaPaca-2 pancreatic cancer cells (FIGS. 8b and 8c).

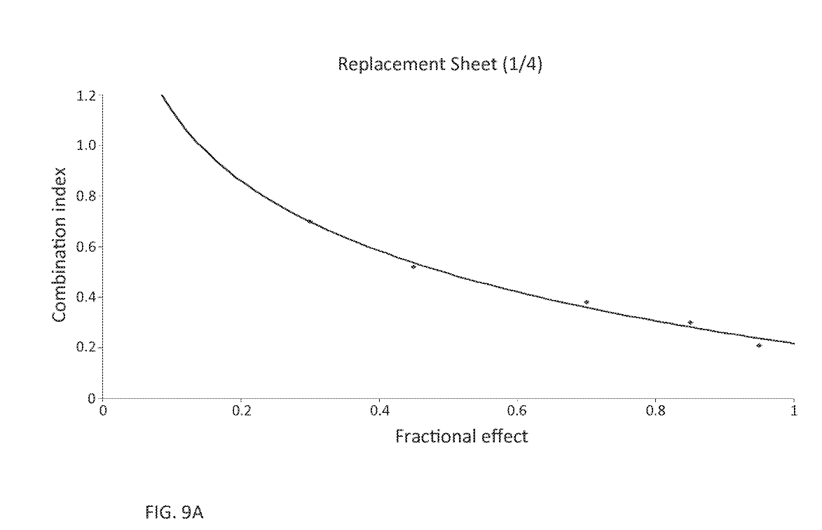

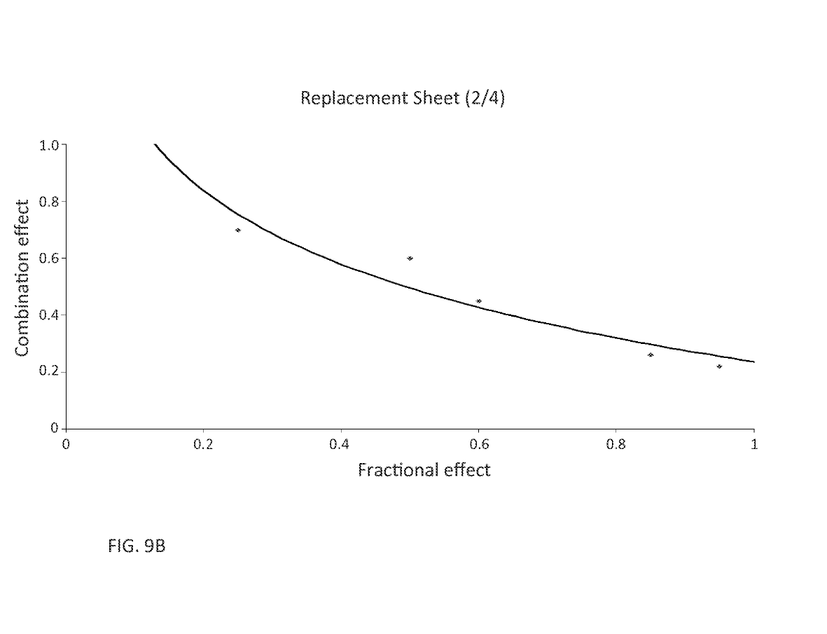

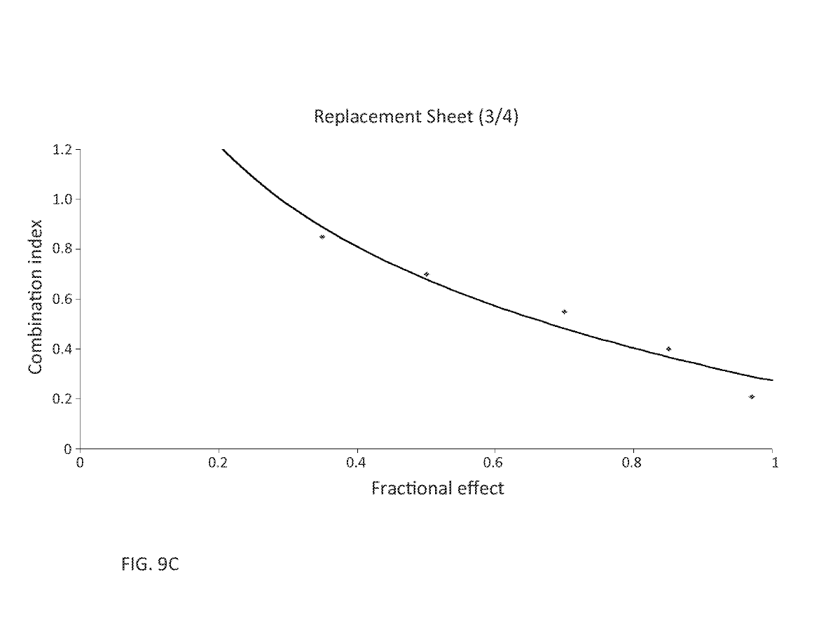

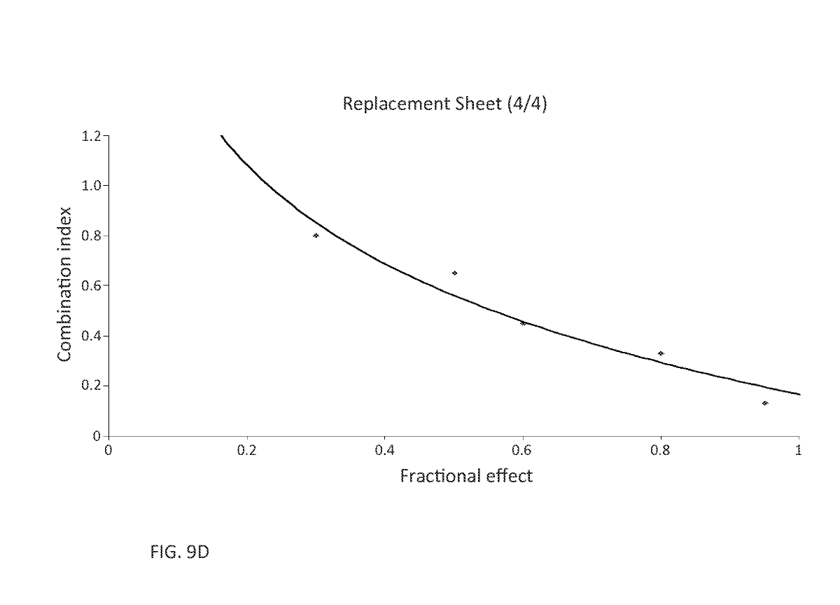

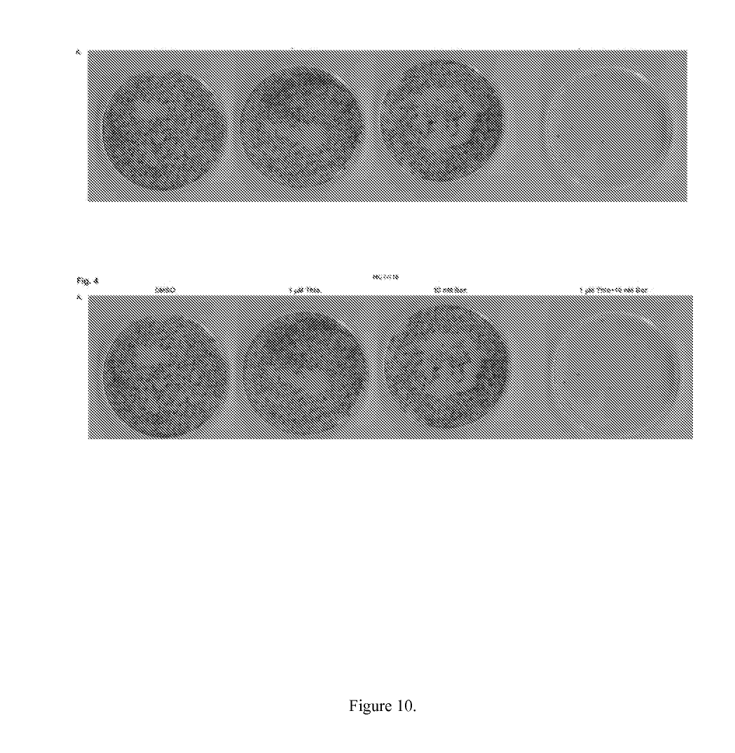

To quantitatively validate the synergistic nature of the interaction between thiostrepton and bortezomib, the cell viability after single and combination drug treatments was examined by using the Chou-Talalay median-effect equation method. CI values below 1 indicate a synergistic anti-proliferative effect, and the CI range values for the combined treatments with thiostrepton/bortezomib in HCT-116, Mia-Paca-2, MDA-MB231 and PA-1 human cancer cell lines were 0.1 to 0.8 (FIG. 9a-9d) for fractional effect corresponding to 0.3 to 0.9, suggesting a strong synergistic effect. The long-term effects of combination treatment with thiostrepton and bortezomib were assessed by clonogenic assay. The colony formation in HCT-116 colon and MDA-MB 231 breast cancer cells treated with combination of these drugs for 24 hr was suppressed (FIGS. 10a and 10b) more than 10-fold (data not shown) suggesting that this combination affects long-term survival of cancer cells.

### Example 5

Animal Breast Tumor Model Studies In Vivo with Nanoparticle Micelle Coated Compositions of Thiostrepton in Combination with Bortezomib

### Materials

MDA-MB-231-luc-D3H2-LN, human lymph node-derived metastatic mammary gland adenocarcinoma (Caliper Lifescience) were maintained in MEM media (Mediatech) supplemented with 10% FBS (Atlanta Biological), 1% 100× non-essential amino acids (Gibco), 1% 200 mM NaPyruvate (Gibco) and 75 μg/mL Zeocin (Invitrogen). The 1,2-distearoyl-sn-glycero-3-phosphoethanolamine-N-[methoxy(polyethylene glycol)-2000] (DSPE-PEG₂₀₀₀-MeO) and 1,2-dioleoyl-sn-glycero-3-phosphoethanolamine-N-(lissamine rhodamine B sulfonyl) (Rhodamine-DOPE) was purchased from Avanti Polar Lipids. Thiostrepton (from Streptomyces azureus, 90% purity) was obtained from Sigma and bortezomib was purchased from Millennium pharmaceutics. Purified proteasomes and the YVAD-amc proteasome substrate were purchased from Enzo.

The nanoparticle micelle coated composition of thiostrepton was prepared according to the procedures outlined in Example 1. Final solutions contained 1 mM of thiostrepton contained in 3 mM of DSPE-PEG₂₀₀₀-MeO. For fluorescence labeling, DOPE-Rhodamine was incorporated at the chloroform stage at a 1:100 molar ratio to DSPE-PEG₂₀₀₀-MeO. Sizes and Zeta potentials of nanoparticles (in PBS) were analyzed by dynamic light scattering, using a 5 mW 633 nm laser angled at 90° to the sample.

### Animal Maintenance and Tumor Xenograft Experiments

Animals were maintained and treated in accordance with the Animal Care and Use Committee of UIC. Tumor models were prepared by implanting 1×10⁶ of MDA-MB-231-luc cells suspended in 50 μL of 1:1 PBS/Matrigel into each flank of 4-week old male athymic mice (Taconic). Treatment began once tumors reached sizes of 100 mm³. After completion of the dosing schedule, animals were sacrificed and tumors were removed. Tumors were frozen in liquid N₂ and kept at −80° C. until further analysis.

### Biodistribution of Nanoparticle Micelle Coated Compositions Containing Thiostrepton

Rho-labeled micelle-thiostrepton nanoparticles (1 mM in PBS) were injected via the tail-vein into MDA-MB-231 tumor-bearing animals (1 cm³ per tumor) at a dose of 40 mg thiostrepton/kg. At 4 hr, 17 hr and 24 hr post-injection, animals were sacrificed, and organs were removed and imaged using the Xenogen IVIS system for rhodamine-fluorescence distribution (excitation wavelength of 570 nm, emission wavelength of 620 nm). Background autofluorescence was eliminated using a spectral unmixing algorithm provided by Living Image software and photon flux per organ was measured again with Living Image software.

Treatment of Xenograft Models with Nanoparticle Micelle Coated Compositions Containing Thiostrepton in Combination with Bortezomib

Animals bearing tumors were randomized into groups of 4-5, where groups were administered with either 30 mg/kg of thiostrepton encapsulated in micelles (2 mM thiostrepton/6 mM _{DSPE-PEG}₂₀₀₀-MeO in PBS, 200-300 μL i.v.), 0.5 mg/kg Bortezomib (in saline, 50 μL per injection i.p.) or with both thiostrepton 30 mg/kg and bortezomib 0.5 mg/kg. Treatments were performed once every two days (3 times a week), during which tumor volumes were monitored with calipers (l×w×h=volume, mm³). Animals were also weighed once a week. After 5 treatments, animals were sacrificed and tumors removed. Removed tumors were divided into two pieces, one piece was fixed in 10% formalin (for 24 hr, followed by storage in 70% EtOH solution) and the other was frozen in liquid N₂ and mechanically homogenized (Fisher, Polytron) in IP lysis buffer (20 mM HEPES, 1% Triton X-100, 150 mM NaCl, 1 mM EDTA, 1 mM EGTA, 100 mM NaF, 10 mM Na₄P₂O₇, 1 mM Na₃ VO₄, 0.2 mM PMSF).

For bioluminescence imaging, animals were injected intraperitonially with 100 mg luciferin/kg 10 min before animals were anaesthetized under isoflurane. Animals were then imaged for tumor-associated bioluminescence (IVIS, Xenogen), where relative units of bioluminescence were quantified as flux (photon/sec) and analyzed with Living Image software.

### Immunohistochemical and Western Blot Analysis of Tumor Tissue

Formalin-fixed tumor tissues were embedded in paraffin and sliced to 4 μm-thick sections. Sections were deparaffinzed by submerging sample-containing slides into a series of solvents of decreasing hydrophobicity. Heating samples in 10 mM citric acid (pH 6) was used for antigen retrieval. After blocking with 3% H₂O₂ in MeOH, and further blocking in normal goat serum in PBS, slides were treated with primary anti-anti-cleaved caspase-3 or control IgG antibodies at concentrations of 1:100, in 1% BSA/PBS. Biotinylated secondary antibody treatment and Avidin HPR treatment was carried out according to the Vectastain ABC kit manual (anti-rabbit, Vector Labs), followed by staining with DAB (Sigma, D-0426). Samples were further counterstained with hematoxylin (Vector Labs) before dehydration in a series of solvents and mounted with Permount (Fisher) mounting agent. Slides were analyzed on a Zeiss Apotome microscope. For western blot analysis, protein concentrations of tumor homogenates were measured by the Bio-Rad Protein Assay, after which protein separation was performed on 8% or 12% SDS-PAGE gels. Separated proteins were then transferred onto PVDF membranes (Millipore) and immunoblotted with specific antibodies against cleaved PARP (h-250, Santa Cruz) and β-actin (Sigma).

### Proteasome Activity Assay

Homogenized tumor lysates were measured for protein concentration with the Bio-Rad Protein assay. For the proteasome activity assay, 40 μg of protein per tumor were assayed according to the manual instructions (Millipore). Briefly, to each reaction was added tumor lysates (40 μg, ˜10 μL), 10× assay buffer (10 μL, 250 mM HEPES, pH 7.5, 5 mM EDTA, 0.5% NP-40, 0.01% SDS w/v), Ac-YVAD-amc substrate (10 μL, 0.5 mM) and H₂O (˜70 μL). Assay mixtures were incubated at 60° C. for 2 hr, after which solutions were examined for fluorescence at excitation wavelength of 340 nm and emission wavelength of 505 nm. Values are represented as a % of the proteasome activity of non-treated tumors.

To assay proteasome inhibitor activities of proteasome inhibitors purified proteasomes, (0.5 μg, 1 μg/μL) were pre-incubated with inhibitors bortezomib (5 nM, 1 μL of 0.5 μM in DMSO) and/or thiostrepton (20 μM, 1 μL of 2 mM) in 10× assay buffer (10 μL) and H₂O (88.5 μL) for 30 min at room temperature. Ac-YVAD-amc substrate (10 μL, 0.5 mM) was then added and the assay solution was incubated at 60° C. for 2 hr, after which solutions were examined for fluorescence at excitation wavelength of 340 nm and emission wavelength of 505 nm. Values are represented as a % of the proteasome activity of non-inhibited proteasomes.

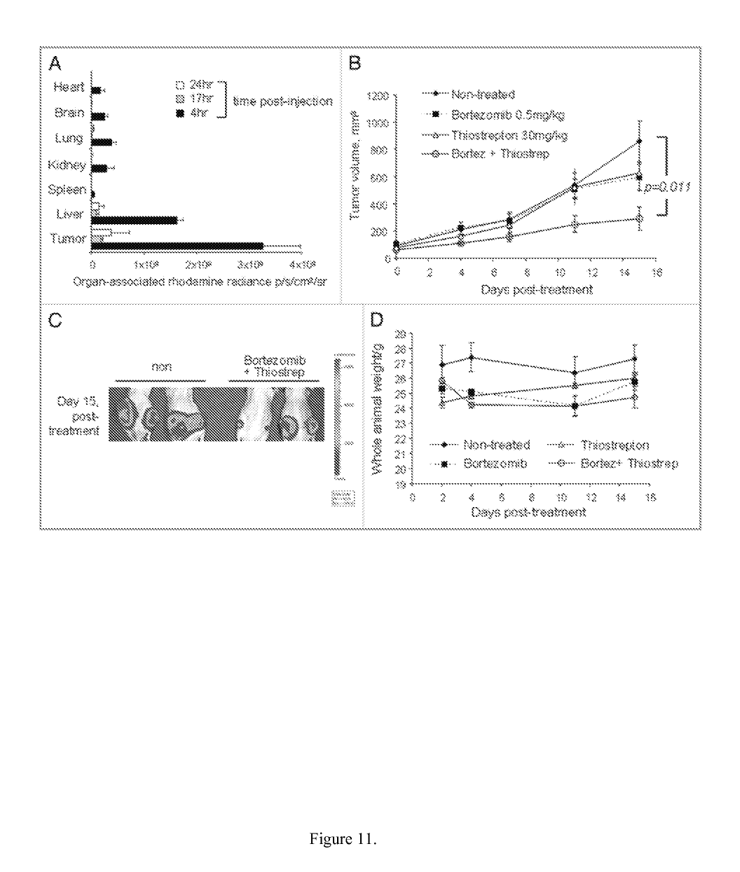

Following administration of fluorescently-tagged thiostrepton-micelle compositions into animals bearing tumors, fluorescence within the tumor was highest at 4 hr post-administration, whereas by 17 hr, the tumor-associated fluorescence had predominately diminished (FIG. 11a). This was demonstrative that micelle-thiostrepton was able to reach tumors at high concentrations after intravenous injection, upon which its anticancer effects can be exerted. To determine the combinatory effect of bortezomib and thiostrepton, animals bearing triple-negative MDA-MB-231-luc solid breast tumor xenografts were administered with either bortezomib (0.5 mg/kg), micelle-encapsulated thiostrepton (30 mg/kg) or a combination of the two. These doses for the single treatments were chosen for their near lack of effect on inhibiting tumor growth when administered alone. The in vitro synergic effect of this combination for inducing apoptosis in the MDA-MB-231 cell line was previously described. After a dosing schedule of 5 injections (15 days, 2-3 injections per week), tumors treated with a combination of proteasome inhibitors appeared to be significantly smaller in size, compared to non-treated tumors and to those treated with single drugs (FIG. 7 B). Bortezomib and micelle-thiostrepton-treated tumors progressed in size at a rate similar to that of non-treated tumors, although a slight reduction in tumor growth was observed after repeated treatment (FIG. 11b). After completion of the treatment schedule, non-treated tumors had increased in size (compared to the first day of treatment) by 10-fold, bortezomib and thiostrepton-treated tumors had increased in size by 6-fold, whereas tumors treated with a combination of the drugs only increased in volume by 3-fold (FIG. 11b). This is depicting the enhanced effect of treating solid tumors with a combination of proteasome inhibitors. The implanted tumor cells also stably express luciferase, therefore live imaging of tumor-associated bioluminescence also allowed the determination of tumor cell viability. Bioluminescence data correlates with that of the tumor growth curve, where non-treated tumors expressed higher amounts of luciferase, compared to tumors treated with a combination of proteasome inhibitors (FIG. 11c). After the treatment schedule, only animals treated with a combination of proteasome inhibitors showed weight loss, probably a result of both the inhibition of tumor growth (less tumor mass) and the slight increase in toxicity from combination treatment. However, it is likely that any toxicity is far from lethal, as the weight loss was a maximum of 5% (FIG. 11d).

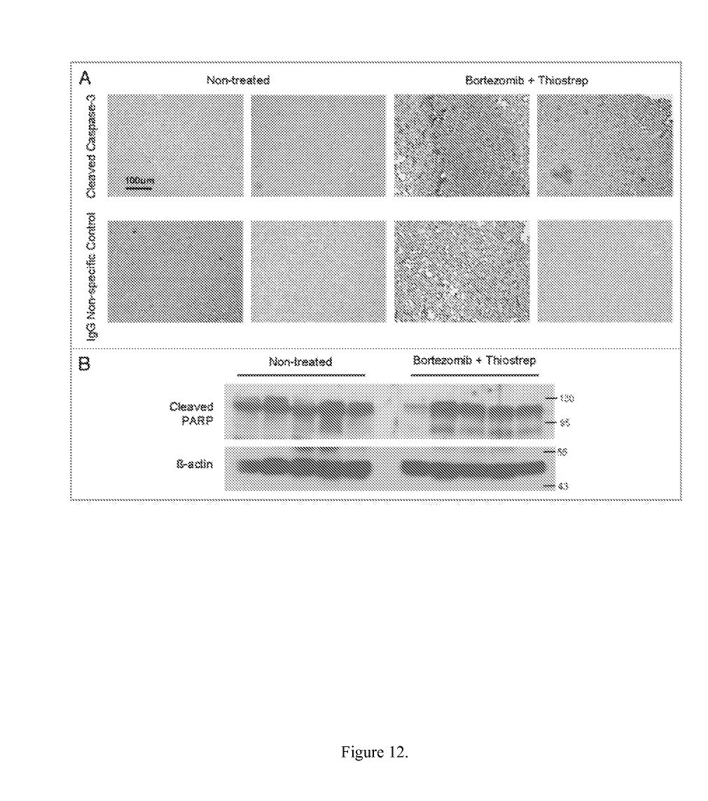

Upon completion of the dosing program, tumors were harvested and examined for markers of apoptosis Immunohistochemical analysis on individual tumors demonstrated enhanced induction of cleaved caspase-3 in combination-treated tumors, compared to non-treated tumors (FIG. 12a). Furthermore, individual homogenized tumor samples also showed increased promotion of cleaved-PARP, another apoptotic marker, in combination-treated tumors compared to non-treated tumors (FIG. 12b). The suppression of tumor growth by combination treatments is likely associated with induction of cell death within the tumor cells. Tumor homogenates were also examined for differences in proteasome activities of tumor-associated proteasomes. Homogenized tumor lysates were incubated with the fluorogenic peptide YVAD-amc, a proteasome substrate that is cleaved by the caspase-like site within the proteasome.

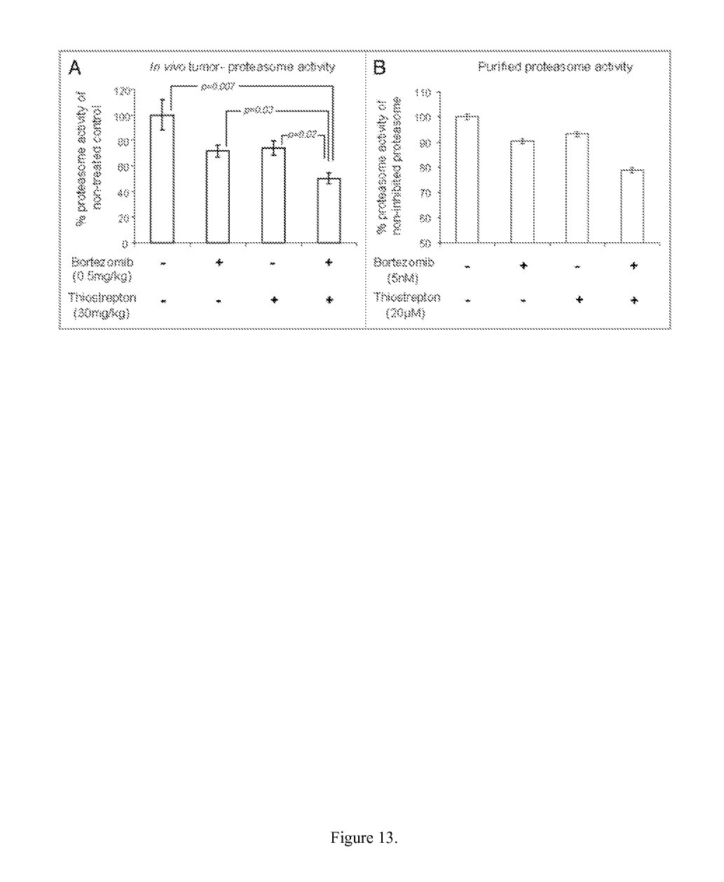

Tumor-associated proteasomes from non-treated tumors maintained the highest activity (FIG. 13a). Contrastingly, tumors treated with a combination of the proteasome inhibitors bortezomib and thiostrepton exhibited impaired proteasome function, as demonstrated by the reduced abilities of tumor-associated proteasomes to cleave the fluorogenic substrate (FIG. 13a). Furthermore, single drug-treated tumors also showed reduction in proteasome substrate cleavage, however to a lesser extent than tumors treated with the combination of complementary inhibitors. The decrease in proteasome activity in combination-treated tumors is likely a result of proteasome inhibition by both bortezomib and thiostrepton, which ultimately leads to the suppression of tumor growth. Similar effect is observed when the combination of such complementary proteasome inhibitors is used to inhibit the activity of purified proteasomes (FIG. 13b). Bortezomib and thiostrepton alone only slightly hindered the effect of purified proteasomes to cleave YVAD-amc substrates, whereas the effect of proteasome obstruction was more apparent when used in combination (FIG. 13b). The results demonstrate the complementary effect of thiostrepton plus bortezomib on inhibition of breast xenograft tumor growth in vivo. The anticancer effect of combination of thiostrepton and bortezomib is superior to their individual treatments and has potential as a therapy against solid tumors.

### Example 6

Animal Liver Tumor Model Studies In Vivo with Nanoparticle Micelle Coated Compositions of Thiostrepton in Combination with Bortezomib

The efficacy of nanoparticle micelle coated compositions containing thiostrepton in inhibiting tumor growth in DEN/PB hepatocellular carcinoma mouse model was evaluated. The enhanced anti-cancer effect of the combination treatment of the thiostrepton composition with bortezomib was also determined in this liver cancer animal model.

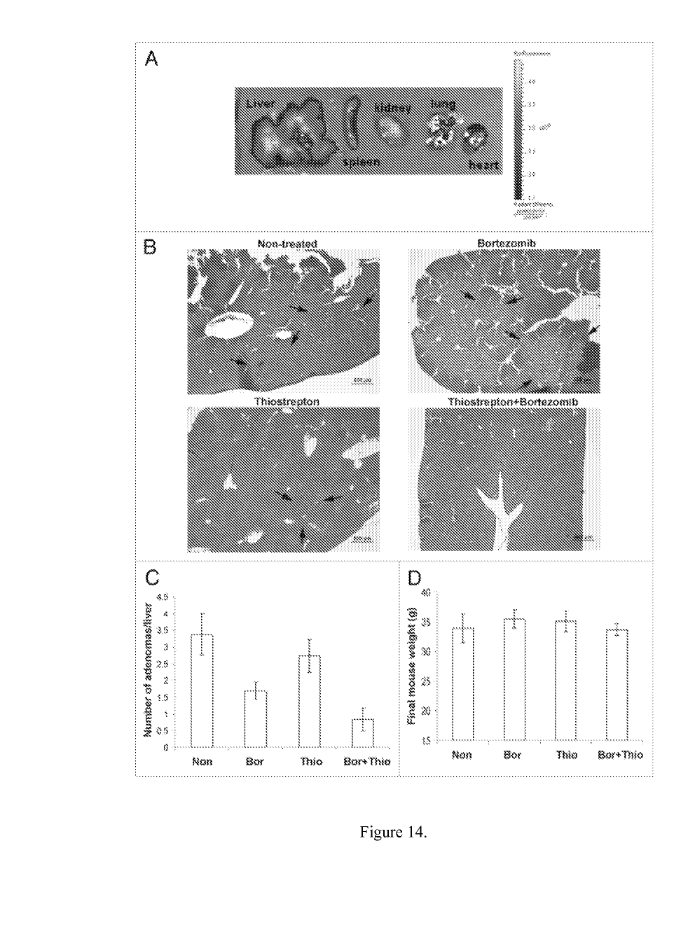

Spontaneous induction of HCC was achieved by intraperitonial injection of the carcinogen diethylnitrosamine (DEN) into 2-day-old neonatal BALB/C mice. Carcinogenesis was further induced by continuing the mice on phenobarbital (PB)-doped drinking water (1 mg/mL). Six weeks after the exposure to the DEN/PB protocol, animals were separated into treatment groups of, a) non-treated control, b) thiostrepton-micelle only 30 mg/kg i.v., c) bortezomib only 0.5 mg/kg, i.p. and, d) combination of thiostrepton 30 mg/kg and bortezomib 0.5 mg/kg. To study the pharmacodistribution of thiostrepton-micelle nanoparticles in mouse liver cancer models, two animals were administered with fluorescently-labeled thiostrepton-micelles (30 mg/kg, i.v.) for examination of the pharmacodistribution of such a nanomedicine in endogenous liver cancer models. Four hours post-injection, over 90% of micelle-associated fluorescence was accumulated into the livers of treated animals (FIG. 14a). This occurrence is likely to be associated with the diffusion of nanoparticle species through the fenestrations within the sinusoidal capillaries of the liver vasculature. This phenomenon renders nanoparticle drug delivery of anti-cancer drugs to liver cancers advantageous, due to the organ-specificity of the delivery process. As a treatment schedule, animals were treated once every other day (3 times a week) for 5 weeks before sacrifice, after which livers were removed and examined for visible tumor growth (FIG. 14b).

Compared to control animals, animals treated with the thiostrepton-micelle composition alone, bortezomib alone or a combination of both thiostrepton-micelle composition and bortezomib had livers exhibiting a lower number of visibly apparent tumor formations. Animals treated with only the thiostrepton-micelle composition had 20% fewer number of visible tumors; animals treated with only bortezomib had 50% fewer number of visible tumors; and animals treated with a combination of the thiostrepton-micelle composition and bortezomib had a 75% fewer number of visible tumors (compared to non-treated control animals). Animal livers were then fixed in 4% paraformaldehyde, embedded in paraffin wax and the right lobe of embedded livers were sectioned and stained with H&E for quantitative analysis of carcinoma formation. Livers obtained from animals treated with either thiostrepton-micelle alone, bortezomib alone or with a combination of the two exhibited diminished numbers of adenocarcinomas and carcinomas, compared to livers from non-treated animals (FIG. 14c). More specifically, treatment with either thiostrepton-micelle or bortezomib alone resulted in a suppression of the number of adenomas, compared to livers from non-treated animals, but a combined treatment with both thiostrepton-micelle composition and bortezomib resulted in a greater suppression of tumor growth, by 75% (FIG. 14c).

Thiostrepton and bortezomib are both proteasome inhibitors, and their complementary anti-cancer action are demonstrated herein in xenograft cancer models. By way of explanation that in no way should be construed to limit the applicability of the compositions disclosed herein, such an effect is most likely due to the differential binding of thiostrepton and bortezomib to separate sites of the proteasome, resulting in the inhibition of both chymotrypsin and caspase-like cleavage sites. The body weights of treated animals did not decrease significantly compared to that of non-treated animals, providing indication to the low toxicity profiles of the single treatments using either the thiostrepton-micelle composition or bortezomib alone and of the thiostrepton-micelle composition/bortezomib combination treatment (FIG. 14d).

The data provides a further demonstration of the anti-cancer efficacy of the thiostrepton-micelle composition as a nanomedicine against liver cancer. Co-administration of bortezomib with the thiostrepton-micelle composition is able to inhibit further the formation of adenocarcinomas and carcinomas in an endogenous hepatocellular carcinoma mouse model. It is likely that the inhibition of FOXM1, an oncogenic transcription factor over-expressed in hepatocellular cancer by both proteasome inhibitors plays a role in their anticancer effect. Previous studies in such an HCC model have shown similar effects on tumor inhibition by another FOXM1-inhibitor, ARF-peptide, through suppressing the oncogenic activity of FOXM1. Not only does the thiostrepton-micelle composition also effects suppression of FOXM1 expression in liver tumor models, but also the liver-targeting biodistributional properties of the thiostrepton-micelle composition contributes to its low toxicity as exposure to non-cancerous tissues is minimized (FIG. 14a). This highlights the clinical potential of thiostrepton-micelle compositions in combination with bortezomib to be adopted as a treatment option for solid tumors, particularly for liver cancers.

### Terminology and Definitions

The terminology used herein is for the purpose of describing particular embodiments only, and is not intended to be limiting. With respect to the use of substantially, any plural and/or singular terms herein, those having skill in the art can translate from the plural as is appropriate to the context and/or application. The various singular/plural permutations may be expressly set forth herein for the sake of clarity.

The terms “nanoparticle micelle coated composition containing thiostrepton,” “micelle-encapsulated thiostrepton” and “thiostrepton-micelle composition” have the same meaning and are used interchangeably as described and used herein.

The term “m/m” refers to molecule per molecule, or mole per mole.

The abbreviations, i.v. and i.p. refer to intravenous (or intravenously) and intraperitonial (or intraperitonially), respectively.

Terms used herein are intended as “open” terms (e.g., the term “including” should be interpreted as “including but not limited to,” the term “having” should be interpreted as “having at least,” the term “includes” should be interpreted as “includes but is not limited to,” etc.).

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into subranges as discussed above.

A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiments or examples disclosed, but that the present invention will include all embodiments falling within the scope of the appended claims.

What is claimed is:

1. A composition comprising:
(a) thiostrepton; and
(b) a micelle-forming lipid,
wherein the thiostrepton is encapsulated inside a nanoparticle comprising the micelle-forming lipid.

2. The composition according to claim 1, wherein the micelle-forming lipid comprises at least one member selected from the group consisting of
(i) 1,2-distearoyl-sn-glycero-3-phosphoethanol-amine-N-[methoxy(polyethylene glycol)-X], wherein X=1000 MW, 2000 MW, 5000 MW, 10000 MW, 20000 MW, or 40000 MW;
(ii) 1,2-distearoyl-sn-glycero-3-phosphoethanol-amine-N [amino(polyethylene glycol)-X], wherein X=1000 MW, 2000 MW, 3400 MW, 5000 MW or 10000 MW;
(iii) 1,2-distearoyl-sn-glycero-3-phosphoethanol-amine-N-[maleimide-(polyethylene glycol)-X], wherein X=1000 MW, 2000 MW, 3400 MW, 5000 MW, or 10000 MW;
(iv) 1,2-distearoyl-sn-glycero-3-phosphoethanol-amine-N-[hydroxyl(polyethylene glycol)-X], wherein X=5000 MW;
(v) 1,2-distearoyl-sn-glycero-3-phosphoethanol-amine-N-[azido(polyethylene glycol)-X], wherein X=3400 MW or 5000 MW;
(vi) 1,2-distearoyl-sn-glycero-3-phosphoethanol-amine-N-[silane(polyethylene glycol)-X], wherein X=3400 MW;
(vii) 1,2-distearoyl-sn-glycero-3-phosphoethanol-amine-N-[succinyl(polyethylene glycol)-X], wherein X=2000 MW;
(viii) 1,2-distearoyl-sn-glycero-3-phosphoethanol-amine-N-[carboxyl(polyethylene glycol)-X], wherein X=2000 MW, and combinations thereof.

3. The composition according to claim 1, wherein the micelle-forming lipid comprises at least one member selected from the group consisting of
(i) Poly(lactic-co-glycolic-acid) (PLGA), polyethyleneglycol (PEG);
(ii) Poly(ethylene oxide)-Poly(α-benzyl L-aspartate);
(iii) Poly(ethylene oxide)-block-poly(L-aspartate);
(iv) Poly(ethylene oxide)-β-p(ε-caprolactone);
(v) Polyethylene Glycol-Diacyl lipid;
(vi) Hydrolyzed polymer of epoxidized soybean oil; and combinations thereof.

4. The composition according to claim 1, wherein the micelle-forming lipid comprises 1,2-distearoyl-sn-glycero-3-phosphoethanol-amine-N-[methoxy(polyethylene glycol)-2000].

5. The composition according to claim 4, wherein the molar ratio of thiostrepton to the 1,2-distearoyl-sn-glycero-3-phosphoethanol-amine-N-[methoxy(polyethylene glycol)-2000] is about 1:3.

6. The composition according to claim 4, wherein the thiostrepton concentration ranges from about 0.2 mM to about 10.0 mM.

7. The composition according to claim 1, further comprising an additional chemotherapeutic agent.

8. The composition of claim 7, wherein the additional chemotherapeutic agent resides outside the nanoparticle comprising the micelle-forming lipid.

9. The composition of claim 8, wherein the additional chemotherapeutic agent comprises bortezomib.

10. A method of treating a subject having a solid tumor, said method comprises:
administering to said subject a therapeutically-effective amount of a nanoparticle micelle coated composition of thiostrepton.

11. The method of claim 10, wherein administering to said subject comprises injecting intravenously said nanoparticle micelle coated composition of thiostrepton.

12. The method of claim 10, wherein the solid tumor comprises at least one member selected from the group consisting of liver cancer, breast cancer, pancreatic cancer, ovarian cancer, osteoscarcoma, colon cancer, prostate cancer, and lung cancer.

13. The method of claim 10, wherein the nanoparticle micelle coated composition of thiostrepton comprises a concentration of thiostrepton from about 0.2 mM to about 10.0 mM.

14. The method of claim 10, wherein the nanoparticle micelle coated composition of thiostrepton comprises 1,2-distearoyl-sn-glycero-3-phosphoethanol-amine-N-[methoxy(polyethylene glycol)-2000 as a micelle-forming lipid.

15. The method of claim 14, wherein the administering to said subject a nanoparticle micelle coated composition of thiostrepton further comprises administering an additional chemotherapeutic agent.

16. The method of claim 15, wherein the additional chemotherapeutic agent comprises bortezomib.

17. A pharmaceutical composition for treating a subject having a solid tumor, comprising a thiostrepton-micelle composition.

18. The pharmaceutical composition of claim 17, wherein the thiostrepton-micelle composition comprises:
(a) a therapeutically effective amount of thiostrepton, and
(b) a micelle-forming lipid comprising 1,2-distearoyl-sn-glycero-3-phosphoethanol-amine-N-[methoxy(polyethylene glycol)-2000], wherein the molar ratio of thiostrepton to the micelle-forming lipid is about 1 to 3.

19. The pharmaceutical composition of claim 17, further comprising a therapeutically effective amount of bortezomib.
